(19) United States
(12) Patent Application Publication
(10) Pub. No.: US 2025/0081965 A1
(43) Pub. Date: Mar. 13, 2025

(54) METHOD OF USING/APPLYING A KERATIN HYDROLYSIS PEPTIDE SOLUTION UPON COFFEE PLANTS TO INCREASE FRUITING YIELD AND WEIGHT

(71) Applicant: CH Biotech R&D Co., Ltd., Nantou City (TW)

(72) Inventors: Iou-Zen CHEN, Taipei (TW); Chuan LEE, Taichung (TW); Pei-Chun LIAO, New Taipei City (TW); Yu-Yi WU, Taichung (TW); Feng-Ju HO, Pingtung (TW); Tsai-Yu YANG, Taichung (TW)

(73) Assignee: CH Biotech R&D Co., Ltd., Nantou City (TW)

(21) Appl. No.: 18/531,687

(22) Filed: Dec. 7, 2023

(30) Foreign Application Priority Data
Sep. 13, 2023 (TW) 112134952

(51) Int. Cl. A01N 37/46; A01P 21/00; C07K 14/465

(52) U.S. Cl. CPC A01N 37/46; A01P 21/00; C07K 14/465

(57) ABSTRACT

Present invention teaches the method of using a keratin hydrolysis peptide (“KHP”) solution to increase the coffee production. By selectively choosing specific weights of feathers and water, and treating the mixture, though one embodiment does not have water mixed in, to a high-temperature high-pressure hydrolysis process, the resulting solution is confirmed to contain at least 253 peptides and then applied to the leaf surface of coffee plants at different growth stages. Optionally, the KHP solution can be diluted by water, as taught in the specification, before applying to the leaf surface of the coffee plants.

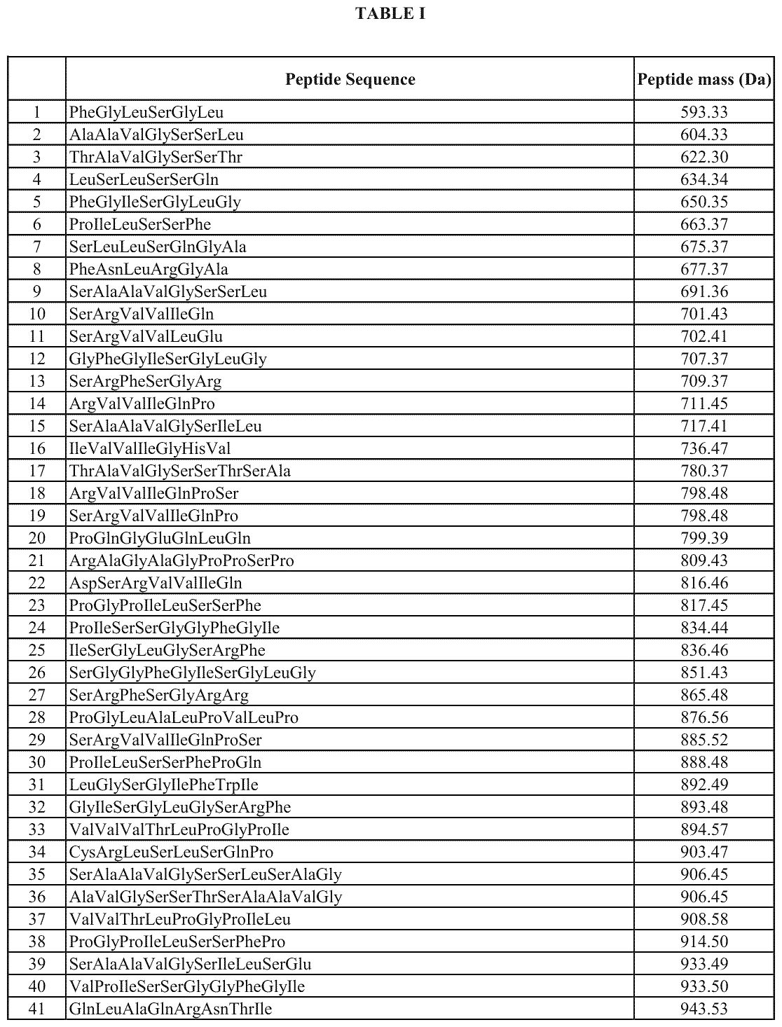

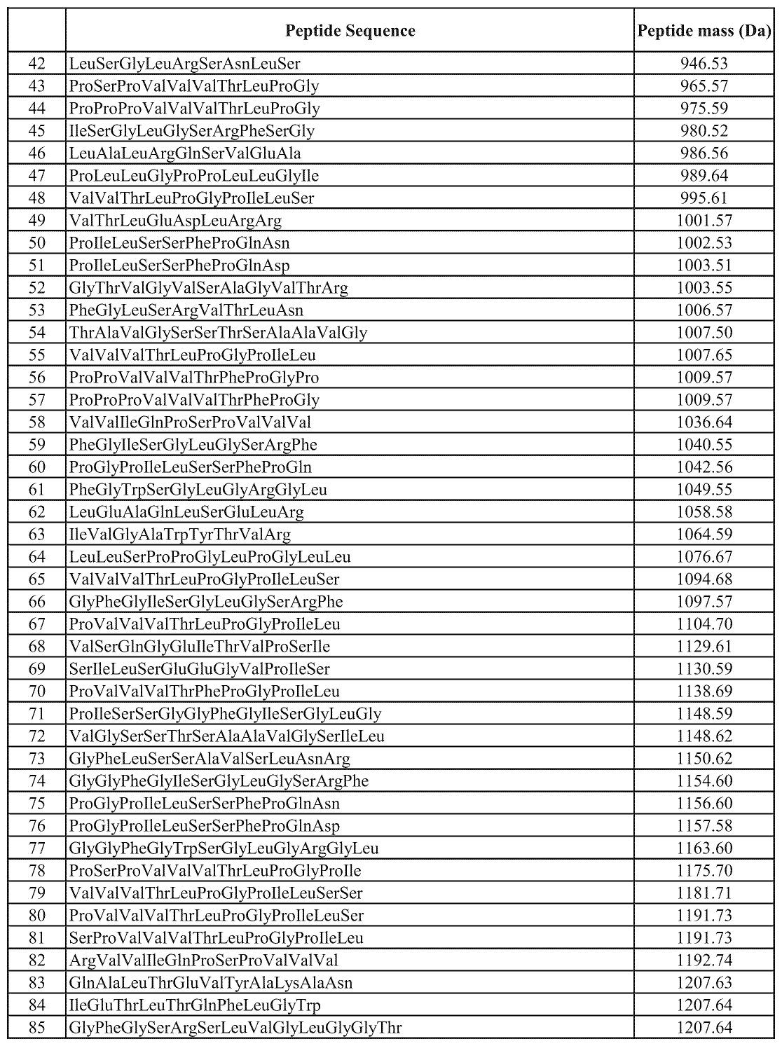

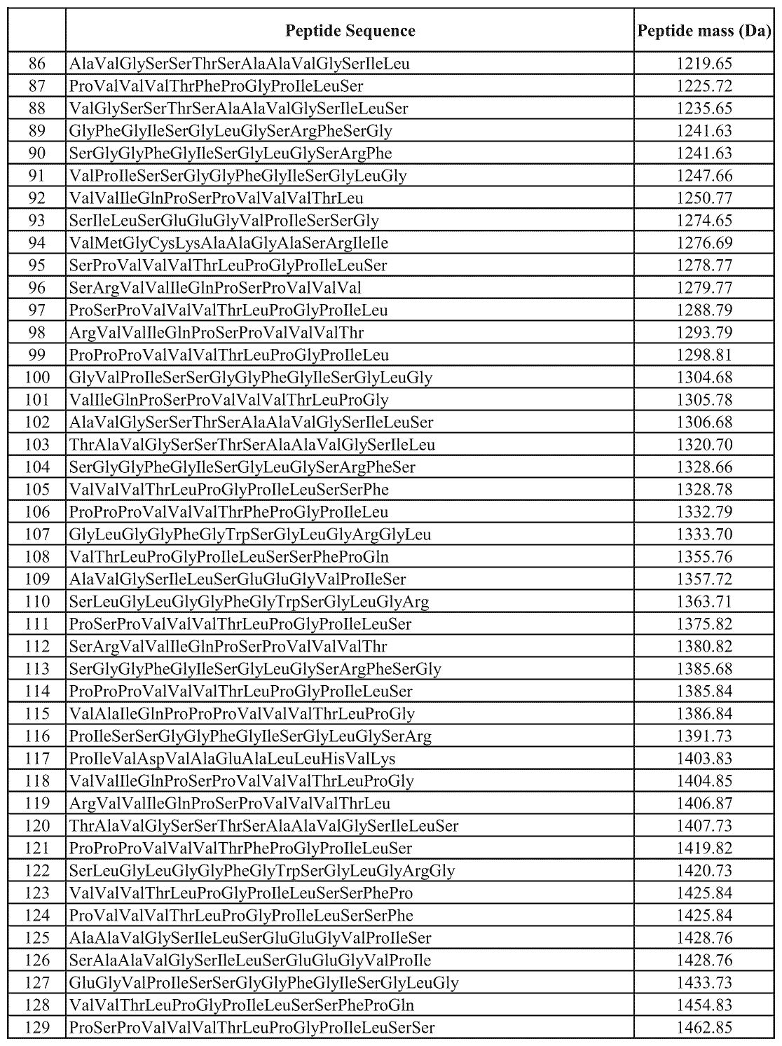

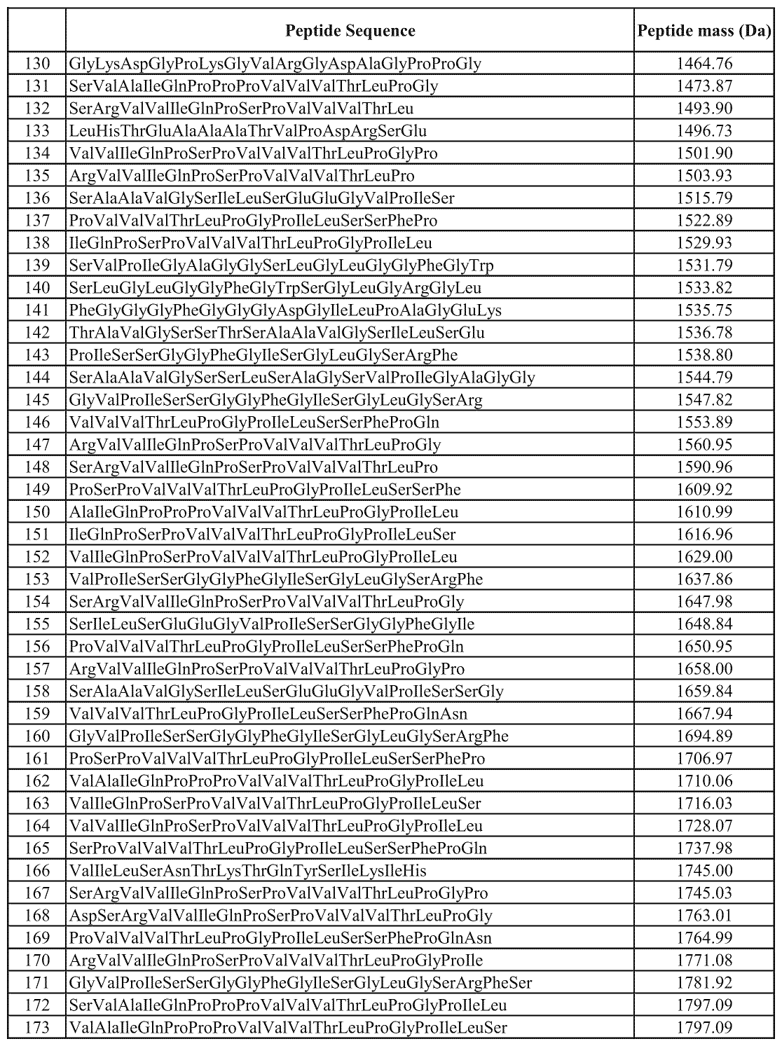

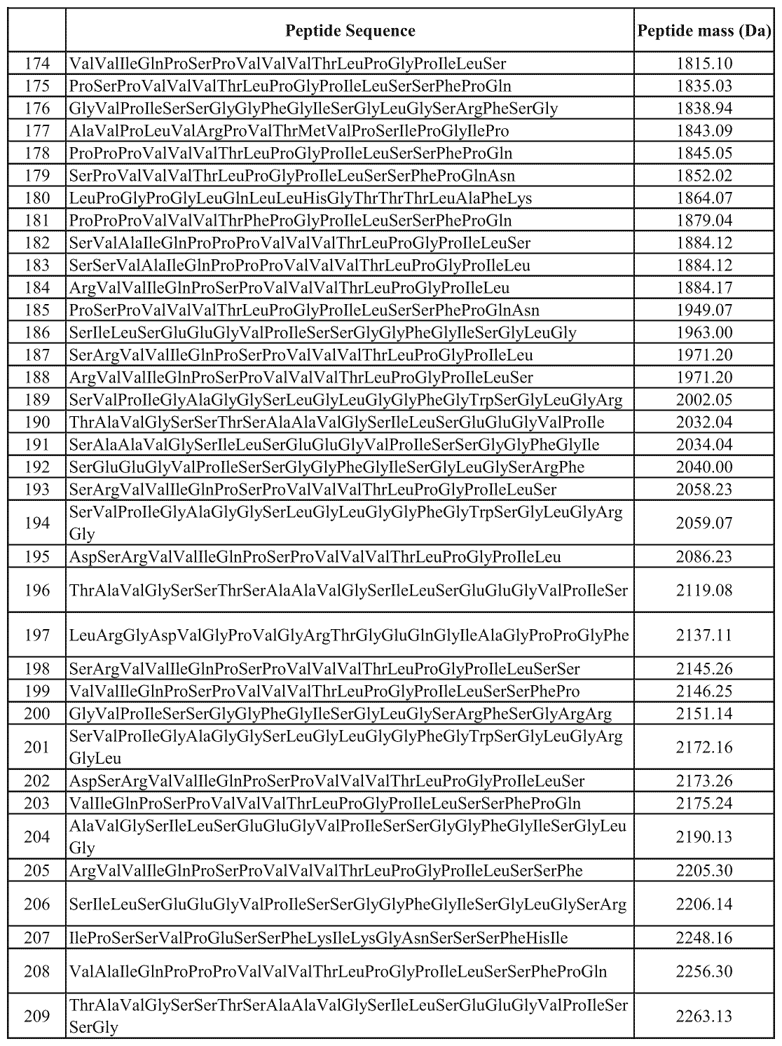

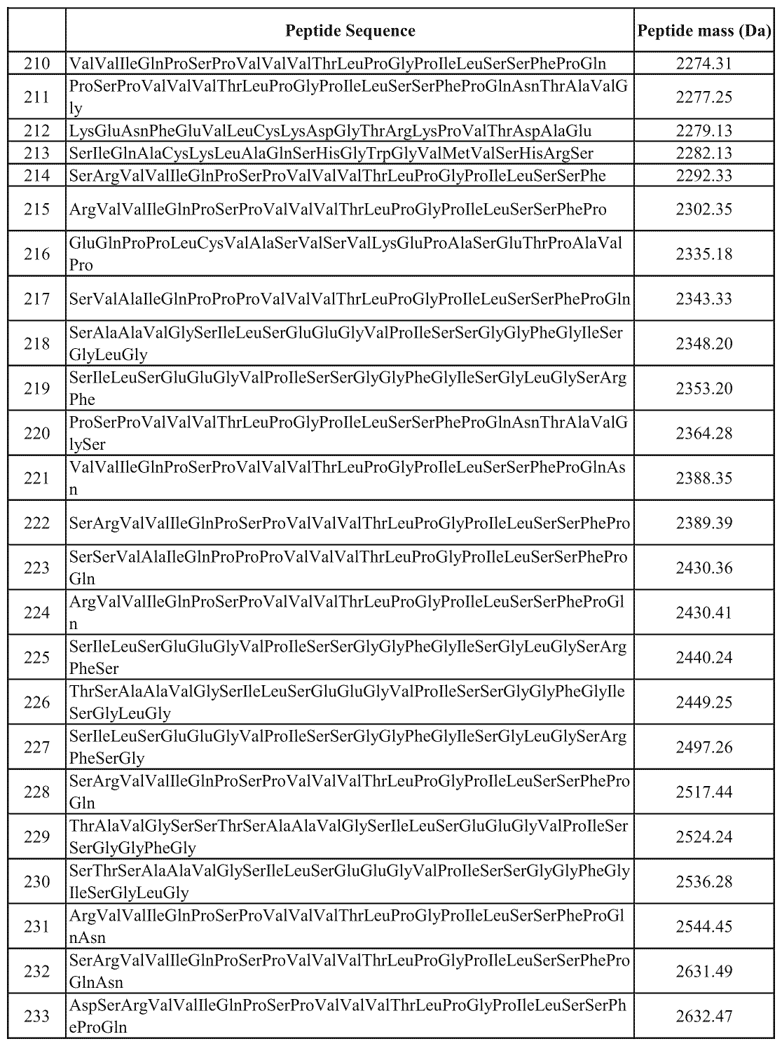

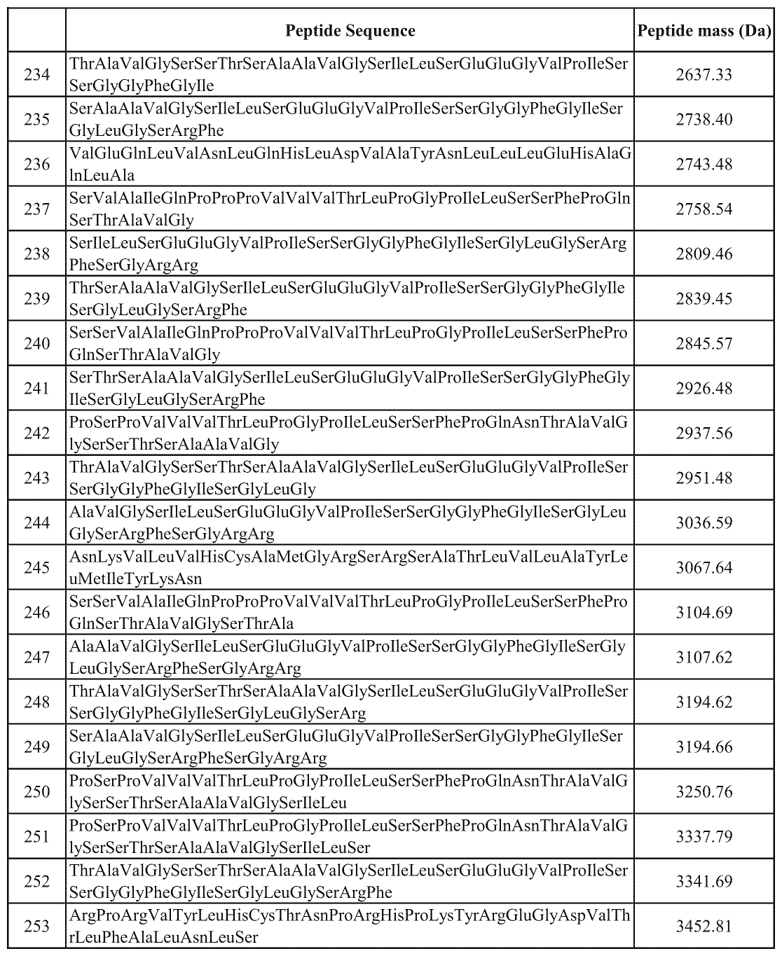

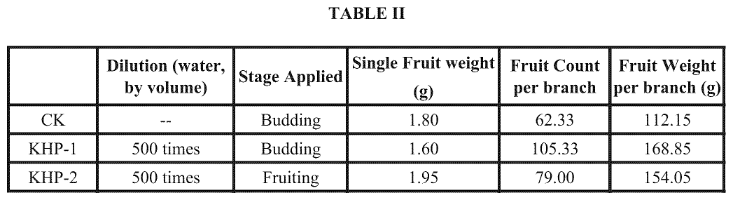

### PRIORITY CLAIM TO FOREIGN APPLICATION

Applicant hereby makes priority claim to a Taiwan application, number 112134952, having the Taiwan filing date of Sep. 13, 2023.

### BACKGROUND OF THE INVENTION

Present invention disclosed and claimed the method and application of a keratin hydrolysis peptide (KHP) solution to coffee trees/plants, increasing the fruiting weight and yield.

The KHP solution is made by a hydrolysis process using feathers and water, with one version of the solution using feathers only, via a high-temperature and high-pressure process, resulting in a solution that has many beneficial applications in the fields of horticulture, agriculture and potentially other farming businesses.

Hydrolyzed keratin has long been used to strengthen hairs, reduce breakage, and minimize damages. Some form of chemical process is used to break down the large protein molecule of the hydrolyzed keratin so that it can penetrate the hair cuticles and do its magic.

Coffee planting, harvesting and drinking is one of the most important economy activities dating back hundreds of years in many places of the world. Many countries, such as Brazile, Colombia and Vietnam, among others, heavily rely on the export of coffee to boost their GDPs. Better coffee fruiting weight and yield will certainly add to financial benefits of the farmers in the coffee-exporting countries, as well as consumers worldwide.

The Australian Climate Institute's 2016 report, A Brewing Storm: The Climate Change Risks to Coffee, pointed out that, if nothing to done to alleviate the problems, the worldwide coffee production will be reduced by 50% by 2050. By 2100, when the temperature rise reaches 4.8° C., all wild and natural coffee plants will go extinct.

Consequently, it is urgent that people need to work on solutions to alleviate the impact of climate change that may lead to the growth and production of coffee plants, and work on solutions to reduce the susceptibility of coffee growth/production to the ever-changing climate, as well as short-term or local weather variations.

Adverse weather conditions usually lead to poor growth and sub-standard coffee production/yield. As such, the inventors of present application work on a method of using a keratin hydrolysis peptide (KHP) solution to coffee trees/plants that can produce higher fruiting weight and yield, under the same growth conditions as limited by available land area and in the client environment currently faced by the people.

Among the known ways to deal with the coffee production issue, most rely on the use of adding fertilizers to the soil in which coffee plants are grown. For example, the published Chinese patent of CN109400291A came up with a fertilizer for applying to the leaf surface during spring/summer seasons. The CN109400291 patent mainly used animal based amino acids to mix other ingredients and then goes through fermentation process to obtain a fertilizer that is shown to increase coffee yield.

Another published patent CN11317977B adopted a different approach to increase production yield: selectively choosing certain root growth of coffee plants for grafting to an already-grown plant, resulting in better flowering/blossoming and thus production yield.

The present application takes a completely different route by using the keratin subject that is widely available in bird feathers. It is well known that feathers of domestic birds/fowls contain 85-91% keratin, 13-15% organic nitrogen, 1.6-2% organic sulfur, as well as other materials. The high keratin content has drawn many prior researches that work to break down, by enzyme, chemical reagents, or fermentation process, into peptides, amino acids and other smaller molecules that can be used for animal feeds, plant fertilizers, and cultivation bases.

Around 2019, Nurdiawati, et al, came up with a hydrolysis process, by mixing feathers and water (generally at a rate of 1:3) for treatment of a high-temperature and high-pressure (setting of 160° C., 6.1 kg/cm², or setting of 180° C. and 9.2 kg/cm² for a duration of 30 minutes) that obtained a solution. The keratin solution is then mixed with other fertilizers containing nitrogen, potassium and other minerals before applying to plants.

The keratin hydrolysis solution is shown to boost the growth of Pogostemon cablin and Vigna radiata, as reported by Nurdiawati et al., 2019.

Under the aegis of CH Biotech, inventors of present application experimented and worked on the method of employing a high-temperature and high-pressure hydrolysis process, materially different from Nurdiawati, to break down the keratin in feathers, and to form a hydrolyzed peptide (“KHP”) solution that can be applied to coffee plants, suitable for increasing the fruiting weight and yield, increasing the more cost-effective coffee farming and harvesting.

### SUMMARY OF THE INVENTION

The invention has two embodiments to create the KHP solution, where one version contains the water:feather weight ratio of 1:1.5, before mixing and placed into a sealed contained to go through the hydrolysis process. The second version does not add water, but simply uses feathers.

The first embodiment of the KHP solution is made by the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to leaf surface of the coffee plant.

To confirm the 253 peptides, the inventors used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The method of using a keratin hydrolysis peptide (KHP) solution stated above is to apply to the leaf surface of the coffee plant at the growth stages of early budding and blooming.

The method of using a KHP solution is further diluted with water by volume at the ratio of 1:250-1,000 and is then sprayed to the coffee leaves' surface at the growth stages of early budding and blooming.

A second embodiment of the method of using a keratin hydrolysis peptide (KHP) solution comprising the steps of:
a. Preparing the KHP solution by preparing and putting 70 kg of feathers whose content is 46% water in a sealed container;
b. hydrolyzing the mixture in the container with a temperature and pressure setting of 180° C. and 13 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to the leaf surface of the coffee plant.

The same reference to BIOPEP-UWM database is made to confirm the bioactive peptides.

The method of using a keratin hydrolysis peptide (KHP) solution stated above is to apply to the leaf surface of the coffee plant at the growth stages of early budding and fruiting.

The method of using a KHP solution is further diluted with water by volume at the ratio of 1:250-1,000 and is then sprayed to the coffee leaves' surface at the growth stages of early budding and blooming.

To prove the efficacy of the method of increasing coffee fruiting weight and yield, the inventors went through experiments in the field and presented the result herein.

The inventors chose the coffee species of Coffea Arabica, a popular planting species in Taiwan coffee farming industry.

Using the second embodiment, the inventors diluted the KHP solution with 500 times of water, by volume, for applying to coffee plants' leaf surface at different stages. There are two groups: KHP-1 group is applied with the KHP solution at early budding stage. KHP-2 group is applied with the solution at fruiting stage. A check group (noted as CK) is not applied with the KHP solution.

During different growth stages, the same water/nutrient conditions were given to the three (3) groups. As shown in Table II, the coffee fruit count and weight show higher numbers in the two groups applied with KHP solutions than the check group.

Based upon the actual experiments, it also appears that the method has the highest effectiveness when the solution is applied at the early budding stage, as the average per-branch count increased went from 62 (in the Check group) to 105 (in the KHP-1 group).

The average per-branch yield also has significant increase, from 112 g (in the Check group) to 168 g (in the KHP-1 group).

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

Table II shows the comparisons of coffee yield when the two groups, KHP-1 and KHP-2, having KHP solution applied at different stages according to the disclosure of present application.

### DETAILED DESCRIPTION OF THE INVENTION

The method of using a keratin hydrolysis peptide (KHP) solution to a coffee plant for the enhancement of the fruiting weight and yield, comprising the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to leaf surface of the coffee plant.

The confirmation of some of the bioactive peptides is further done by referencing the BIOPEP-UWM database.

A method of using a keratin hydrolysis peptide (KHP) solution to coffee plants for the enhancement of fruiting weight and yield, comprising the steps of:
a. Preparing the KHP solution by preparing and putting 70 kg of feathers whose content is 46% water in a sealed container;
b. hydrolyzing the mixture in the container with a temperature and pressure setting of 180° C. and 13 kg/cm² for a duration of 40 minutes;
c. using a mass spectrometer confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to the leaf surface of the coffee plant.

Some of the bioactive peptides are confirmed by referencing the BIOPEP-UWM database.

The method of using a keratin hydrolysis peptide (KHP) solution stated above where the solution is applied to the leaf surface of the coffee plant at the growth stages of early budding and fruiting.

The method of using a KHP solution stated above where the solution is diluted with water by volume at the ratio of 1:250-1,000 and is sprayed to the coffee leaves' surface at the growth stages of early budding and/or fruiting.

In the research and experiment to prove the efficacy of the method as taught herein, the inventors used a species of Coffea Arabica, SL34, which is a common plant species in Taiwan.

Two comparison groups, KHP-1 and KHP-2, are applied with the KHP solution (from the embodiment of 70 kg feathers without water), diluted 500 times with water, at different growth stages: KHP-1 was applied at early budding stage and KHP-2 was applied at fruiting stage.

The same metric acre of the coffee plants are treated with 1,000 liters of water (CK group) and the 2 solutions (KHP-1 and KHP-2).

The field comparison is done by selecting three branches from each group to measure the single fruit weight, fruit count per branch and weight per branch. The average numbers are then calculated. The comparison shows that both the Fruit Count per branch and Weight per branch increased substantially in the two groups where the KHP solution was applied at the designated growth stages.

In terms of Fruit Count per branch, it appears that the KHP-1 group raised the number from 62 (reflected in the CK group) to 105, a 69% increase where the application of the KHP-1 solution was done at the budding stage.

In terms of fruit weight per branch, the KHP-1 has 168.85 g and the KHP-2 has 154.05 g, all far better than the CK group number of 112.15, with the application at budding stage showing better result.

As to single fruit weight, only the KHP-2 group shows increase from 1.80 g to 1.95 g when the application of the KHP-2 solution is done at fruiting stage. The KHP-1 group's 1.60 single fruit weight number could be an outlier point, given that only three branches are selected for the comparison, though the aggregate figures of Fruit Count per branch and Weight per branch show substantial increase as discussed previously.

Overall, the research and experiment show that the method of making the KHP solutions and the application substantially add to the coffee fruits' production yield.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method of using a keratin hydrolysis peptide (KHP) solution to coffee plants for the enhancement of the fruiting weight and yield, comprising the steps of:
a. Preparing the KHP solution by preparing and putting 70 kg of feathers whose content is 46% water in a sealed container;
b. hydrolyzing the mixture in the container with a temperature and pressure setting of 180° C. and 13 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to the leaf surface of the coffee plant.

2. The method of using a keratin hydrolysis peptide (KHP) solution of claim 1 where the solution to applied to the leaf surface of the coffee plant at the growth stage of early budding.

3. The method of using a keratin hydrolysis peptide (KHP) solution of claim 1 where the solution to applied to the leaf surface of the coffee plant at the growth stage of fruiting.

4. The method of using a KHP solution of claim 2 where the solution is diluted with water by volume at the ratio of 1:250-1,000.

5. The method of using a KHP solution of claim 3 where the solution is diluted with water by volume at the ratio of 1:250-1,000.
